(19) United States
(12) Patent Application Publication
(10) Pub. No.: US 2002/0170082 A1
(43) Pub. Date: Nov. 14, 2002

(54) Gene affecting male fertility in plants

(76) Inventors: Marc C. Albertsen, Grimes, IA (US); Tim Fox, Des Moines, IA (US)

Correspondence Address: Patricia A. Sweeney, 1835 Pleasant, West Des Moines, IA, 50265-2334, US

(21) Appl. No.: 09/829,378

(22) Filed: Apr. 9, 2001

Related U.S. Application Data
(63) Continuation of application No. 09/340,684, filed on Jun. 29, 1999.

(51) Int. Cl. A01H 1/02; A01H 5/00

(52) U.S. Cl. 800/274; 800/278

(57) ABSTRACT

Plant genes of the Ms*5126 family affect male fertility. A method for developing a male sterile plant, for plant hybridization purposes, entails genetically inactivating a Ms*5126 gene, so as to impair male fertility.

This application is a continuation of previously filed and co-pending application U.S. Ser. No. 09/340,684 filed Jun. 29, 1999.

### BACKGROUND OF THE INVENTION

There is a need for a reversible genetic system for producing male sterile plants, in particular for autogamous plants. Production of hybrid seed for commercial sale is a large and important industry. Hybrid plants grown from hybrid seed benefit from the heterotic effects of crossing two genetically distinct breeding lines. The commercially desirable agronomic performance of hybrid offspring is superior to both parents, typically in vigor, yield and uniformity. The better performance of hybrid seed varieties compared to open-pollinated varieties makes the hybrid seed more attractive for farmers to plant and therefore commands a premium price in the market.

In order to produce hybrid seed uncontaminated with self-seed, pollination control methods must be implemented to ensure cross-pollination and to guard against self-pollination. Pollination control mechanisms include mechanical, chemical and genetic means.

A mechanical means for hybrid seed production can be used if the plant of interest has spatially separate male and female flowers or separate male and female plants. For example, a maize plant has pollen-producing male flowers in an inflorescence at the apex of the plant, and female flowers in the axiles of leaves along the stem. Outcrossing of maize is assured by mechanically detasseling the female parent to prevent selfing. Even though detasseling is currently used in hybrid seed production for plants such as maize, the process is labor-intensive and costly, both in terms of the actual detasseling cost and yield loss as a result of detasseling the female parent.

Most major crop plants of interest, however, have both functional male and female organs within the same flower, therefore, emasculation is not a simple procedure. While it is possible to remove by hand the pollen forming organs before pollen is shed, this form of hybrid production is extremely labor intensive and expensive. Seed is produced in this manner only if the value and amount of seed recovered warrants the effort.

A second general means of producing hybrid seed is to use chemicals that kill or block viable pollen formation. These chemicals, termed “gametocides,” are used to impart a transitory male-sterility. Commercial production of hybrid seed by use of gametocides is limited by the expense and availability of the chemicals and the reliability and length of action of the applications. A serious limitation of gametocides is that they have phytotoxic effects, the severity of which are often dependent on genotype. Another limitation is that these chemicals may not be effective for crops with an extended flowering period because new flowers that are produced may not be affected. Consequently, proper timing and repeated application of chemicals is required.

Many current commercial hybrid seed production systems for field crops rely on a genetic means of pollination control. Plants that are used as females either fail to make pollen, fail to shed pollen, or produce pollen that is biochemically unable to effect self-fertilization. Plants that are unable to self-fertilize are said to be “self-incompatible” (SI). Difficulties associated with the use of a self-incompatibility system include availability and propagation of the self-incompatible female line, and stability of the self-compatibility. In some instances, self-incompatibility can be overcome chemically, or immature buds can be pollinated by hand before the biochemical mechanism that blocks pollen is activated. Self-incompatible systems that can be deactivated are often very vulnerable to stressful climatic conditions that break or reduce the effectiveness of the biochemical block to self-pollination.

Of more widespread interest for commercial seed production are male-sterility systems that are based on genetic mechanisms of pollen control. These systems are of two general types: (a) genetic male sterility, which is the failure of pollen formation because of mutations in one or more nuclear genes or (b) cytoplasmic-genetic male sterility, commonly referred to as “cytoplasmic male sterility” (CMS), in which pollen formation is blocked or aborted because of an alteration in a cytoplasmic organelle, such as the mitochondria. In both types, there is little impact on female fertility. Genetic male sterility can result from a mutation in one of many genes involved in microsporogenesis. These genes are collectively referred to as male fertility genes. Despite the number of male sterile mutants described in maize, little progress has been made in characterizing the biochemical basis of the genes responsible for male fertility.

Although there are hybridization schemes involving the use of CMS, there are limitations to its commercial value. An example of a CMS system is a specific mutation in the cytoplasmically located mitochondria which can, when in the proper nuclear background, lead to the failure of mature pollen formation. In some instances, the nuclear background can compensate for the cytoplasmic mutation and normal pollen formation occurs through the activity of nuclear “restorer genes” that are specific for a given CMS system. Generally, the use of CMS for commercial seed production involves the use of three breeding lines: a male-sterile line (female parent), a maintainer line which is isogenic to the male-sterile line but contains fully functional mitochondria, and a male parent line. In most crops other than maize, these male-parent lines are referred to as “restorer lines.” They carry the specific restorer genes that imparts male fertility to the hybrid seed. In maize, the presence or absence of restorer genes in the male-parent line is immaterial as CMS-produced hybrid seed is routinely blended with hybrid seed produced from isogenic non-CMS lines.

For crops such as vegetable crops for which seed recovery from the hybrid is unimportant, a CMS system can be used without restoration. For crops for which the fruit or seed of the hybrid is the commercial product, the fertility of the hybrid seed must be restored by specific restorer genes in the male parent or the male-sterile hybrid must be pollinated. Pollination of non-restored hybrids can be achieved by including with hybrids a small percentage of male fertile plants to effect pollination. In most species, the CMS trait is inherited maternally, since cytoplasmic organelles are usually inherited from the egg cell only, and this restricts the use of the system.

CMS systems possess limitations that preclude them as a sole solution to production of male sterile plants. One of the most significant is the limitation on genotypes that can be converted because of the presence of naturally occurring restorer genes in the line being converted. This can limit the range of genotypes available for hybrid production. In another example, one particular CMS type in maize (T-CMS) confers sensitivity to the toxin produced during infection by a particular fungus. Although CMS is still used for a number of crops, CMS systems in certain crops may break down under certain environmental conditions. In other crops, fertility restoration issues limit the desirability of using CMS.

Nuclear (genetic) sterility can be either dominant or recessive. Dominant sterility can only be used for hybrid seed formation if propagation of the female line is possible (for example, via in vitro clonal propagation or by restoration of male fertility via U.S. Pat. No. 5,850,014. Recessive sterility can be used if male-sterile and fertile plants are easily discriminated during the seed increase phase of an inbred. Commercial utility of genetic sterility systems is limited, however, by the expense of clonal propagation and roguing the female rows of self-fertile plants.

Discovery of genes which would alter plant development would be particularly useful in developing genetic methods to induce male sterility because other currently available methods, including detasseling, CMS and SI, have shortcomings.

### SUMMARY OF THE INVENTION

It therefore is an object of the present invention to provide a plant that comprises an endogenous Ms*5126 gene, the expression of which is modified such that the fertility of the male flower is impaired.

It is a further object of the invention to provide a method of affecting male fertility in said plant. The inventive method comprises the steps of (A) modifying plant material to impair the expression of an endogenous Ms*5126 gene and then of (B) obtaining from the plant material a plant that comprises the impaired endogenous gene such that a phenotype of male sterility is expressed.

The expression of an endogenous Ms*5126 gene can be impaired through means such as the sequestration of a messenger RNA (mRNA), corresponding to the gene sequence of interest, by an antisense oligonucleotide or nucleotide analogue complementary to the sequence of the mRNA. In another embodiment, the expression of the endogenous Ms*5126 gene is impaired through “co-suppression.” With co-suppression, a transfected gene construct includes the Ms*5126 gene in sense orientation. Via an unknown trans mechanism, a proportion of the transformants exhibit loss of function of the Ms*5126 gene. In another embodiment of the present invention, the expression of the Ms*5126 gene is impaired via homologous recombination. Gene expression is disrupted when a vector transforms target cells possessing a Ms*5126 gene and stably integrates into the Ms*5126 coding sequence of the host chromosome. In another embodiment, the expression of the Ms*5126 gene is disrupted by using naturally occurring transposable elements, such as the Mutator (Mu) transposable element, to introduce transposable element insertions into the Ms*5126 gene sequence. In yet another embodiment of the present invention, ribozymes is used to impair gene expression by cleaving sense MRNA that encode for a Ms*5126 protein. A phenotype of male sterility results as the cleaved MRNA can not be translated to produce the Ms*5126 protein product required for male fertility. In addition, Ms*5126 gene expression can be impaired via a gene-replacement approach, where the Ms*5126 gene sequence is replaced by a related DNA sequence. That is, the form and/or function of the endogenous Ms*5126 gene is affected by the DNA substitution and results in the male sterile phenotype.

Another aspect of the present invention relates to generating a plant that (1) possesses the impaired endogenous gene and (2) manifests the phenotype of male sterility. Methods of regenerating a plant from a transformed cell or culture vary according to the plant species but are based on known methodology. For example, growth conditions for embryo and shoot formation in various monocotyledon and dicotyledon species are discussed by Binding, REGENERATION OF PLANTS—PLANT PROTOPLASTS 21-73 (CRC Press 1985).

In accordance with the present invention, therefore, a plant is provided that comprises an endogenous Ms*5126 gene, the expression of which is impaired such that male fertility in the plant is impaired. According to the invention, moreover, a method is provided for affecting male fertility in a plant, comprising the steps of (A) modifying plant material to impair the expression of the endogenous Ms*5126 gene; (B) obtaining from the material a plant that comprises the impaired endogenous Ms*5126 gene such that a phenotype of male sterility is expressed.

Other objects, features and advantages of the present invention will become apparent from the following detailed description. It should be understood, however, that the detailed description and the specific examples, while indicating preferred embodiments of the invention, are given by way of illustration only, since changes and modifications within the spirit and scope of the invention may become apparent to those of skill in the art from this detailed description.

### BRIEF DESCRIPTION OF THE DRAWINGS

FIG. 1A and FIG. 1B depict, respectively, a nucleotide sequence and a deduced amino acid sequence for a maize Ms*5126 gene. The nucleotide sequence in FIG. 1A comprise SEQ ID NO:1. The deduced amino acid sequence in FIG. 1B comprises SEQ ID NO:2.

FIGS. 2A and 2B show a comparison of the deduced amino acid sequence of the maize Ms*5126 gene (SEQ ID NO:2) to the chalcone synthase amino acid sequence of Petunia hybrida (petunia) (SEQ ID NO:3) and the stilbene synthase amino acid sequence of Vitis cv. ortis (grape) (SEQ ID NO:4), respectively.

FIG. 3 shows the amino acid sequence of the maize Ms*5126 amino acid sequence (residues 166-175 of SEQ ID NO:2) aligned with the amino acid sequence of the proposed active site for chalcone and stilbene synthases (SEQ ID NO:10). FIG. 3 also shows the biochemical pathway of chalcone and stilbene synthases.

FIG. 4 shows an mRNA (northern) blot analysis that compares the levels of Ms*5126 gene expression in various maize tissues.

FIG. 5 shows a mRNA (northern) blot analysis that charts the levels of Ms*5126 gene expression during the different stages of microsporogenesis in maize.

FIG. 6 is a map of the Ms*5126 gene in maize. The map details the location of translational sites, PCR primers, probes, a restriction site, a mutator (Mu) insertion site and the exon structure of the Ms*5126 gene in the 54-D7 family.

FIG. 7 shows a DNA (Southern) blot analysis of a male sterile maize family (54-D7) hybridized with a 3′ Ms*5126 probe.

FIG. 8 is a chromosome 7 map from the maize genome that shows the chromosomal location of the Ms*5126 gene.

FIGS. 9A, 9B, and 9C present a comparison of the deduced amino acid sequence of the maize Ms*5126 gene (SEQ ID NO:2) to amino acid sequences of male specific chalcone synthase-like proteins in tobacco (SEQ ID NO:5), pine (SEQ ID NO:6) and rice SEQ ID NO:7), respectively.

### DETAILED DESCRIPTION OF PREFERRED EMBODIMENTS

A. Definitions

As used herein, the term “plant” includes references to whole plants, plant organs (e.g. leaves, stems, roots, etc.), seeds and plant cells and progeny of the same. Plant material includes, without limitation, seeds suspension cultures, embryos, meristematic regions, callus tissues, leaves, roots and shoots, gametophytes, sporophytes, pollen and microspores. The class of plants which can be used in the methods of the invention is generally as broad as the class of higher plants amenable to transformation techniques, including both monocotyledonous and dicotyledonous plants. A particularly preferred plant is maize.

In the present description, a condition of “male sterility in a plant” means that the plant produces no pollen, produces only a small fraction of the pollen produced by the wild type, or produces pollen that is substantially incapable of germination. In a preferred embodiment, no pollen at all is produced. In a plant that produces a reduced amount of pollen, the proportion of seeds produced by self-pollination, even if low, may be commercially unacceptable. In a plant that produces pollen that is non-functional, manual detassling may be required, unless the seed producer can be assured that the pollen is totally non-functional prior to use of the male sterile plant in hybrid seed production. In any event, the condition can be ascertained by methodology well known in the art, including such techniques as anther squashes, determining pollen shed and pollen germination tests.

A “structural gene” refers to a DNA sequence that is transcribed into messenger RNA (mRNA) which is then translated into a sequence of amino acids characteristic of a specific polypeptide. “Messenger RNA (mRNA)” denotes an RNA molecule that contains the coded information for the amino acid sequence of a protein. “Protein” refers to a polymer of amino acid residues.

“Expression” refers to the biosynthesis of a gene product. For example, in the case of a structural gene, expression involves transcription of the structural gene into mRNA and the translation of the mRNA into one or more polypeptides.

“Endogenous” indicates some item that is natural or endemic to its surroundings. In particular, it applies here to a class of genetic constructs that is found in the normal genetic complement of the host plant.

“Phenotype” means the physical manifestation of a genetic trait, resulting from a specific genotype and its interaction with the environment.

“Complementary DNA (cDNA)” is a single-stranded DNA molecule that is formed from an mRNA template by the enzyme reverse transcriptase. Typically, a primer complementary to portions of mRNA is employed for the initiation of the reverse transcription. Those skilled in the art can also use the term “cDNA” to refer to a double stranded DNA molecule consisting of said single-stranded DNA molecule and its complementary DNA strand.

A “cloning vector” is a DNA molecule, such as a plasmid, a cosmid, or bacteriophage that has the capability of replicating autonomously in a host cell.

A term “promoter” connotes a region of DNA upstream from the start of transcription that is involved in recognition and binding of RNA polymerase and other proteins to initiate transcription. Tissue-specific, tissue-preferred, cell type-specific, and inducible promoters constitute the class of “non-constitutive promoters.” A “constitutive promoter” is one that is active throughout the life of the plant and under most environmental conditions. An “operator” refers to a DNA molecule that is located toward the 5′ end of a structural gene and that contains a nucleotide sequence which is recognized and bound by a DNA binding protein with either activation or repression function. The binding of a repressor protein with its cognate operator results in the inhibition of the transcription of the structure gene. “Operably linked” refers to a functional linkage between a promoter and a second sequence, where the promoter sequence initiates and mediates transcription of the DNA sequence corresponding to the second sequence. In general, “operably linked” means that the nucleic acid sequences being linked are contiguous and, where necessary to join the two protein coding sequences, are within the same reading frame. “Adjacent” means a region contiguous to the gene or in close proximity thereto, such as approximately within 100-200 Kb of the gene.

In general, “homologues” or “orthologues” of both DNA and protein molecules can be found by reference to “sequence identity”. Sequence identity refers to a comparison made between two molecules using standard algorithms well known in the art. An example of a standard algorithm is the Smith-Waterman algorithm. Waterman, 1984. Bulletin of Mathematical Biology 46:473-500. When sequence identity is used to compare DNA sequences, the open reading frame of SEQ ID NO:1 is used as the reference sequence in defining the percentage of polynucleotides homologous to its length. Similarly, with comparisons among polypeptides, the amino acid sequence of SEQ ID NO:2 is used as the reference sequence in determining the percent identity of homologous polypeptides.

The choice of parameter values for matches, mismatches and inserts or deletions is arbitrary. However, some parameter values have been found to yield more biologically realistic results than others. One preferred set of parameter values for the Smith-Waterman algorithm is set forth in the “maximum similarity segments” approach. See Waterman, supra. In this approach, values of 1 for a matched residue and −⅓ for a mismatched residue are used. Insertions and deletions, x, are weighted as xₖ=1+k/3 where k is the number of residues in a given insert or deletion. Id.

Preferred molecules are those having at least 80% sequence identity to the open reading frame of the DNA sequence. Particularly preferred molecules have at least 90% sequence identity. Even more preferred molecules have at least 95% sequence identity and most preferred molecules have at least 98% sequence identity. Two nucleic acid molecules or proteins are said to “share significant sequence identity” if the two contain regions which possess greater than 85% sequence amino acid or nucleic acid identity.

In the present context, the terms “homologue” and “orthologue” both denote a polypeptide or protein that has an essentially similar activity to a molecule encoded by a Ms*5126 gene, notwithstanding any amino acid substitutions, additions or deletions therein. A homologue is isolated or derived from the same species, an orthologue from another plant species. The amino acids of a homologue or an orthologue may be replaced by other amino acids that are evolutionarily conserved or have similar properties, such as, hydrophobicity, hydrophilicity, hydrophobic moment, charge or antigenicity, and so on.

A “mutation” includes a chemically or genetically induced nucleotide substitution, addition and/or deletion. It also refers to the disruption of gene expression that results from splicing sense mRNA with ribozymes, mRNA sequestration with an antisense sequence, or disruption of the coding sequence by the insertion of a vector, transposon or gene-targeting construct.

A “genomic library” is a collection of clones that contains at least one copy of every DNA sequence in the genome.

B. Isolation of a MS*5126 Gene

(1) Genomic Library Construction:

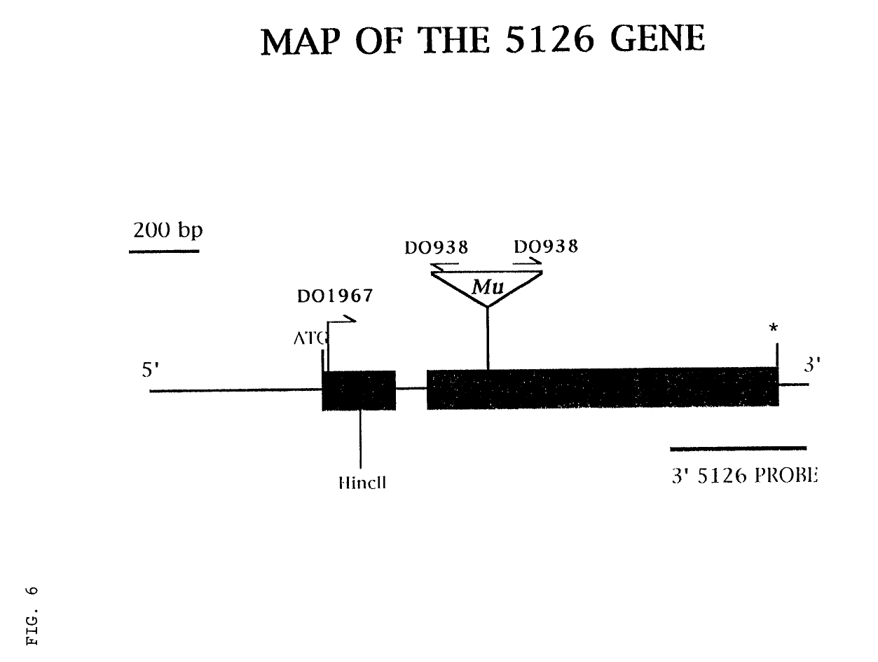

A genomic library can be constructed using methods well known in the art, such as those disclosed in section 5.7.1 of Ausubel, et al. 1995. CURRENT PROTOCOLS IN MOLECULAR BIOLOGY. New York: Wiley Interscience. (“Ausubel”). In accordance with the present invention, for example, a genomic library was constructed from a maize inbred line designated B73. Maize DNA was partially digested with Sau3Al and then cloned into the BamHI site of λ DASH II by Stratagene (LaJolla, Calif.). 1×10⁶ PFU were screened with an EagI fragment from a partial Ms*5126 cDNA. ER1647 (NEB) was used as the host bacterium. Three clones were isolated to homogeneity after three rounds of screening. DNA from these λ clones was isolated using a method reported by Bellomy, et al. 1989. Biotechniques 7:1. The restriction sites of these three clones were mapped. All three clones were identical, spanning approximately 18 Kb. Each clone contained one small, 120 bp intron, approximately 200 bp downstream from the putative translational start site. FIG. 6 refers to a map of the Ms*5126 gene for maize detailing exon structure and translational sites.

(2) RNA Isolation:

RNA can be isolated by means known in the art, such as with the guanidine thiocyanate method described in section 4.2.1 of Ausubel, et al., supra.

(3) mRNA Isolation:

Messenger RNA (mRNA) can be isolated by means known in the art, such as by hybridization to an oligo(dT) column. This method is described more fully in section 4.5.1 of Ausubel, et al., supra.

(4) cDNA Synthesis:

A general method of synthesizing cDNA includes first hybridizing a short oligo(dT) chain to a poly(A) tail at the 3′ end of the mRNA strand. The oligo(dT) acts as a primer for reverse transcriptase which makes a complementary DNA copy of the mRNA strand. Next, RNase H, DNA polymerase I, and DNA ligase are used to synthesize the second DNA strand. RNase H degrades the RNA strand in the hybrid DNA-RNA, DNA polymerase I makes new DNA fragments using the partially degraded RNA fragments as primers, and DNA ligase ligates the new DNA fragments together to make a complete chain. A comprehensive protocol on cDNA synthesis is provided in section 5.5 of Ausubel, et al., supra.

(5) cDNA Library Construction:

A cDNA library can be synthesized using subtraction procedures such as those described, for example, by Diatchenko et al. 1996. Proc. Natl. Acad. Sci. (USA) 93:6025-6030 and, in section 5.9.4-5.9.12 of Ausubel, et al., supra.

More generally, cDNA libraries can be constructed in several ways. For example, in accordance with the present invention, cDNA libraries were made from tassel mRNA from maize stocks of a dominant male sterile mutation (Ms44) and its male fertile sibs (ms44). Both stocks are available from the Maize Stock Center at the University of Illinois. The cDNA libraries were made by Invitrogen (San Diego, Calif.) via a bi-directional cloning method. This bi-directional cloning method involved using a pCD-NAII vector and cloning at BstXI sites. Subtraction procedures were later performed as described by the Subtractor™ I Subtraction Kit for cDNA Probe Generation, Instruction Manual (Invitrogen version 2.3) (“Subtractor”). These procedures included using labeled cDNA from a male sterile dominant library as the driver, and cDNA from an unlabeled male fertile library as the tester. The cDNA library that resulted from this subtraction procedure was designated cDNA library #5.

Another means for creating a cDNA library is exemplified, in accordance with the present invention, by a cDNA library created from maize stock by Stratagene. Stratagene used a Uni-Zap XR directional cloning system (EcoRI to XhoI) to create the cDNA library. This vector is covered by U.S. Pat. No. 5,128,256. 1×10⁶ PFU were screened with an EagI fragment from Ms*5126. ER1647 (NEB) was used as the host bacterium. Ten positive clones were purified to homogeneity. Plasmids were made by in vivo excision of a pBluescript SK(−) phagemid from a Uni-Zap XR vector.

(6) cDNA Probe Construction:

A cDNA probe can be synthesized by means well known in the art. The Subtraction Probe Procedure described in Subtractor, supra, and, discussed in U.S. Pat. No. 5,750,868, the contents of which are incorporated in their entirety, exemplifies a method of making a cDNA probe.

The Subtraction Probe Procedure includes the following steps: Labeled cDNA is first synthesized from the induced (message +) pool of mRNA. The resulting cDNA-RNA hybrid is alkali treated to remove the template MRNA and then hybridized to an excess of photobiotinylated mRNA from pool B (message −). The resulting photobiotinylated RNA/cDNA hybrids are complexed with free streptavidin and removed from the hybridization mixture by selective phenolchloroform extraction. The streptavidin-photobiotinylated nucleic acid complex is extracted leaving the unhybridized (induced) cDNAs behind. The subtracted cDNA probe that results can be used directly in hybridization blots or for screening libraries.

A diagrammatic outline of the Subtraction Probe Procedure is shown below.

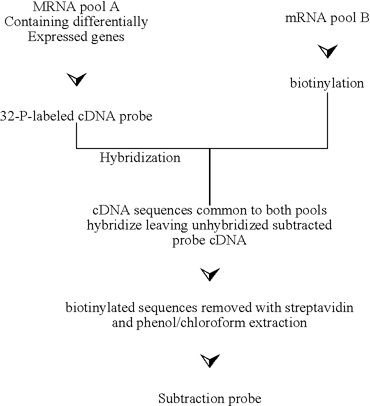

(7) Isolation of Unique Clones from the cDNA Library:

Clones can be isolated via well known screening methods. Protocols exemplifying such methods can be found in section 6 of Ausubel, et al., supra. For example, in accordance with the present invention, clones were isolated randomly from the above referenced subtracted cDNA library #5. Inserts were gel purified and transferred to a nitrocellulose filter and hybridized to random hexamers labeled with p³². Duplicate clones were avoided through cross-hybridization. The isolation procedure resulted in the selection of a Ms*5126 clone from the subtracted cDNA library #5. The Ms*5126 clone was hybridized with non-tassel cDNA to ensure anther specificity.

(8) Sequencing of a Ms*5126 Gene:

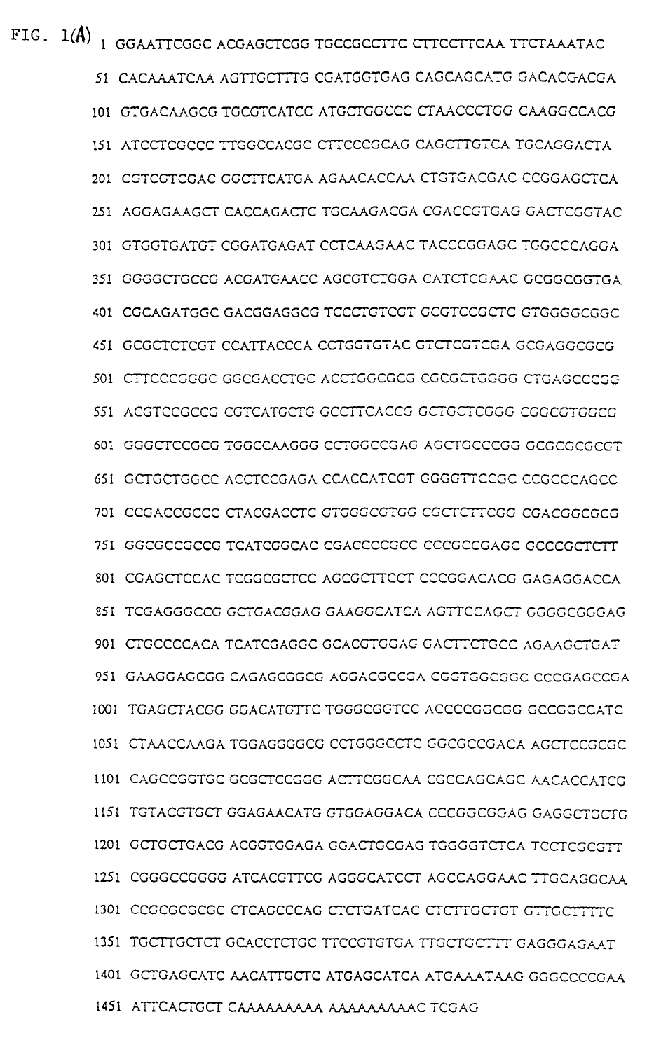

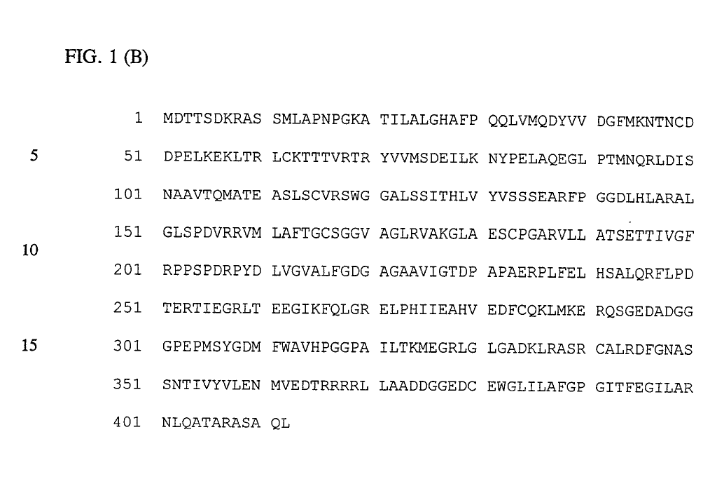

Ms*5126 cDNA can be sequenced using methods well known in the art, such as with procedures disclosed in section 7 of Ausubel, et al., supra. The nucleotide sequence of the Ms*5126 gene from maize is depicted in FIG. 1A and in SEQ ID NO: 1, and the deduced amino acid sequence of a protein encoded by the gene is shown in FIG. 1B and in SEQ ID NO:2. A sequence such as this one can be used in a variety of ways. A partial sequence that is at least about 10 bp in length (although usually more about 15 bp or longer) and extends up to the full-length of the sequence can be used in directing the synthesis of a Ms*5126 gene. In addition, both partial and full-length sequences can be used as probes for the detection of complementary genomic DNA, and as either antisense sequences or co-suppression sequences for inhibiting the expression of a Ms*5126 gene.

C. Characterization of a MS*5126 Gene

(1) Homology of a Ms*5126 Amino Acid Sequence to the Amino Acid Sequences of Chalcone and Stilbene Synthases:

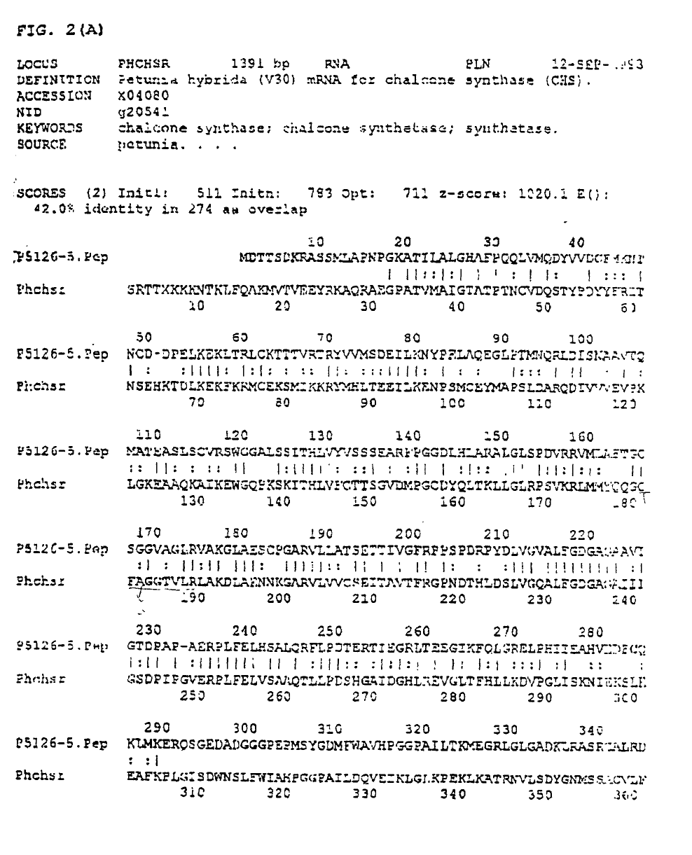

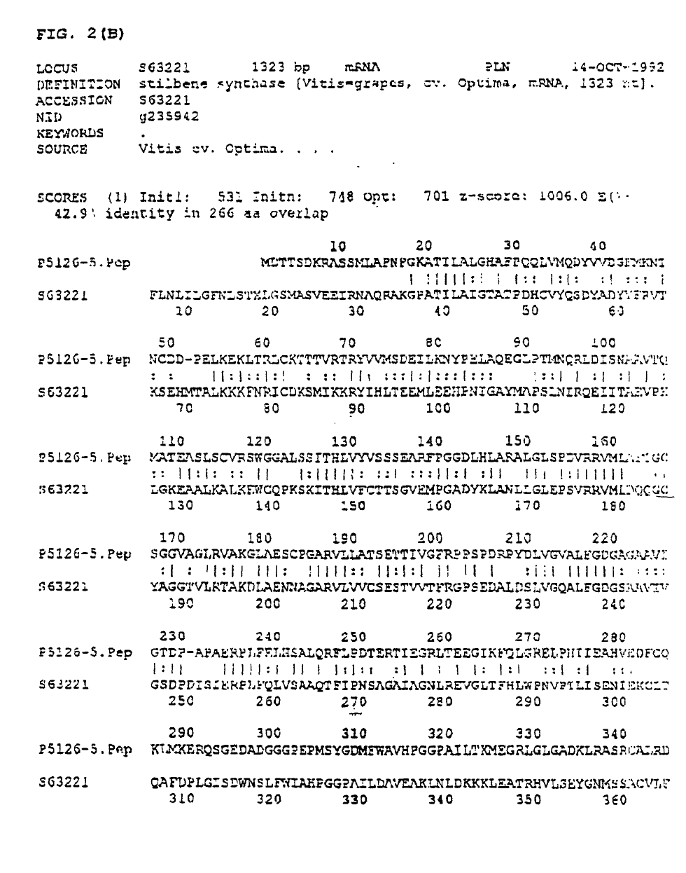

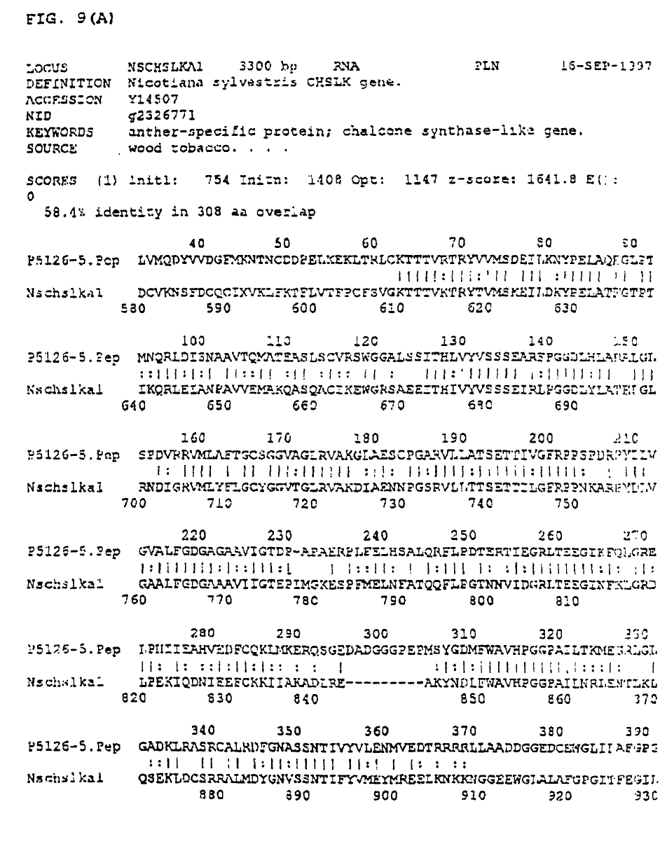

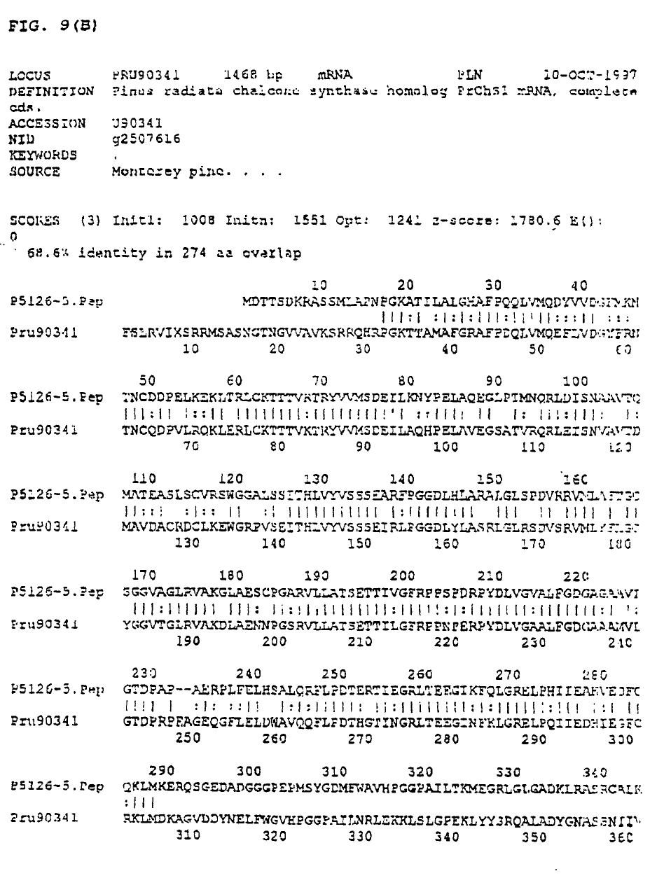

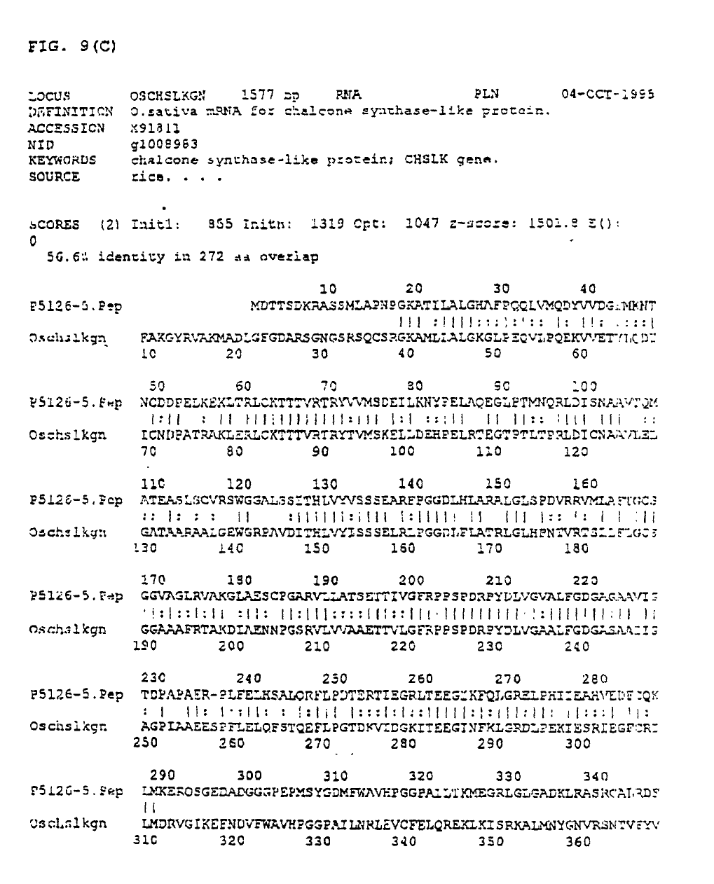

Database searches reveal a significant homology of the amino acid sequence of the maize Ms*5126 gene with amino acid sequences from chalcone and stilbene synthases from a variety of species. For example, FIGS. 2A and 2B point to a 42% identity between the amino acid sequence of the maize Ms*5126 gene and the amino acid sequences of chalcone synthase for petunia and stilbene synthase for grape, respectively. FIG. 9A reveals a 58.4% sequence identity between the amino acid sequence of the maize Ms*5126 gene and the amino acid sequence of a chalcone synthase-like protein for tobacco. FIGS. 9B shows a sequence identity of 68.6% between the amino acid sequence of the maize Ms*5126 gene and the amino acid sequence of a chalcone synthase-like protein for pine. Lastly, FIG. 9C points to a 56.6% sequence identity between the amino acid sequence for the maize Ms*5126 gene and that of a chalcone synthase-like protein for rice.

Homology between the Ms*5126 protein product of maize and chalcone and stilbene synthases (and related homologues) from other plant species suggests that the Ms*5126 protein product may play a regulatory role similar to those played by the synthases. Chalcone synthase is a key enzyme in the flavonoid biosynthesis. The enzyme catalyzes the stepwise condensation of three acetate residues from malonyl-CoA and one residue of 4-coumaroyl-CoA to yield naringenin chalcone. Isomerisation and further substitution of this central intermediate ultimately leads to the production of flavonoids. Flavonoids are secondary metabolites that are known to have a key-function in the pigmentation of flowers and fruit. In addition, flavonoids appear to be involved in the defense against phytopathogens, the protection against UV-light and the induction of nodulation. Flavonoids have also been implicated in the regulation of auxin transport and in resistance to insects.

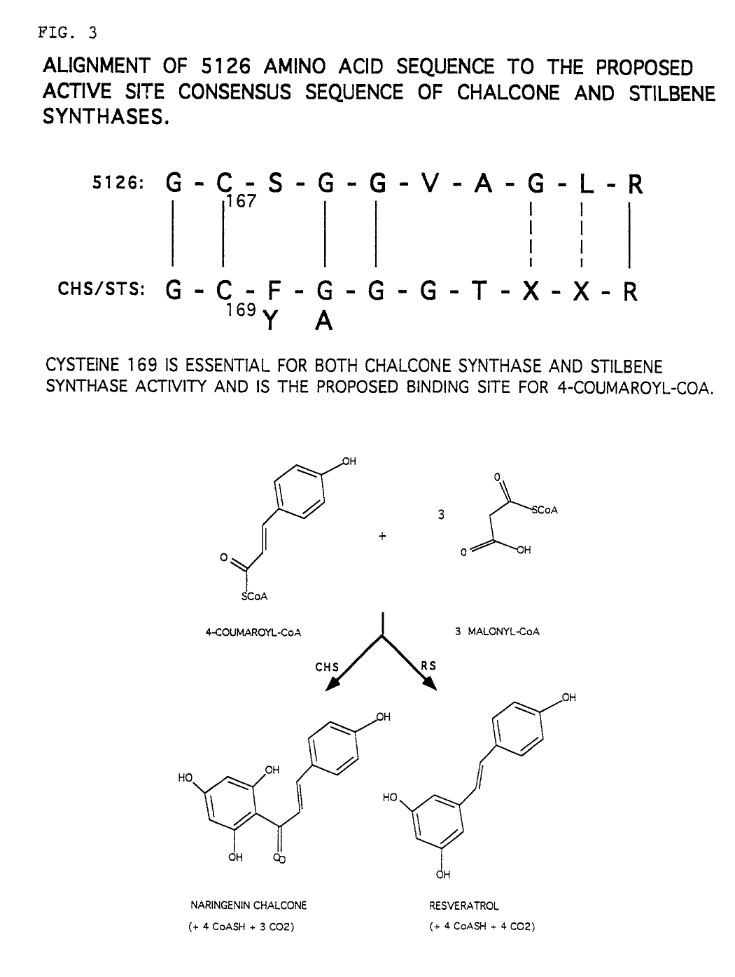

Both chalcone and stilbene synthases utilize the same substrates but produce different end products through a condensation reaction. However, both enzymes have a conserved cysteine residue at amino acid position 169 that, when mutated, abolishes all enzyme activity. This is proposed to be the binding site for 4-coumaroyl-Co-A substrate. Significantly, this cysteine residue is also conserved in the deduced maize Ms*5126 protein at amino acid position 167. FIG. 3 shows the maize Ms*5126 amino acid sequence aligned with the amino acid sequence of the proposed active site of chalcone and stilbene synthases.

(2) Location/Level of Ms*5126 Gene Transcription in Maize Tissue:

A northern blot technique can be performed by methods well known in the art, such as those disclosed in section 4 of Ausubel, et al., supra. A northern blot procedure can be useful in researching gene expression because it can determine the size of the mRNA that is encoded by a gene, the presence and quantity of mRNA in different tissue types, and the level of gene activity during different stages of development. For example, in accordance with the present invention, a northern blot procedure was performed to determine the level of Ms*5126 gene activity during maize microsporogenesis.

A developmental northern was created containing RNA at each stage of microsporogenesis. PolyA+ enriched RNA (mRNA) was isolated from various maize tissues (tassel, green leaf, etoilated leaf and root) and hybridized to a 3′ probe of Ms*5126 that had been isolated from subtraction library #5. PolyA+ enriched RNA was also isolated during fairly discrete stages of maize microsporogenesis, namely: small premeiotic (PREME); large PREME; PREME and Leptotene (LEPT); LEPT and Zygotene (ZYGO); ZYGO and Pachytene (PACHY); Diplotene (DIPLO), meiosis I; meiosis I and Quartet (Q); quartet (Q); quartet and Quartet Release (QR); Early Uninucleate (EU); EU+early Middle Uninucleate (mid uni); mid uni; late mid uni and Late Uninucleate (late uni); and late uni.

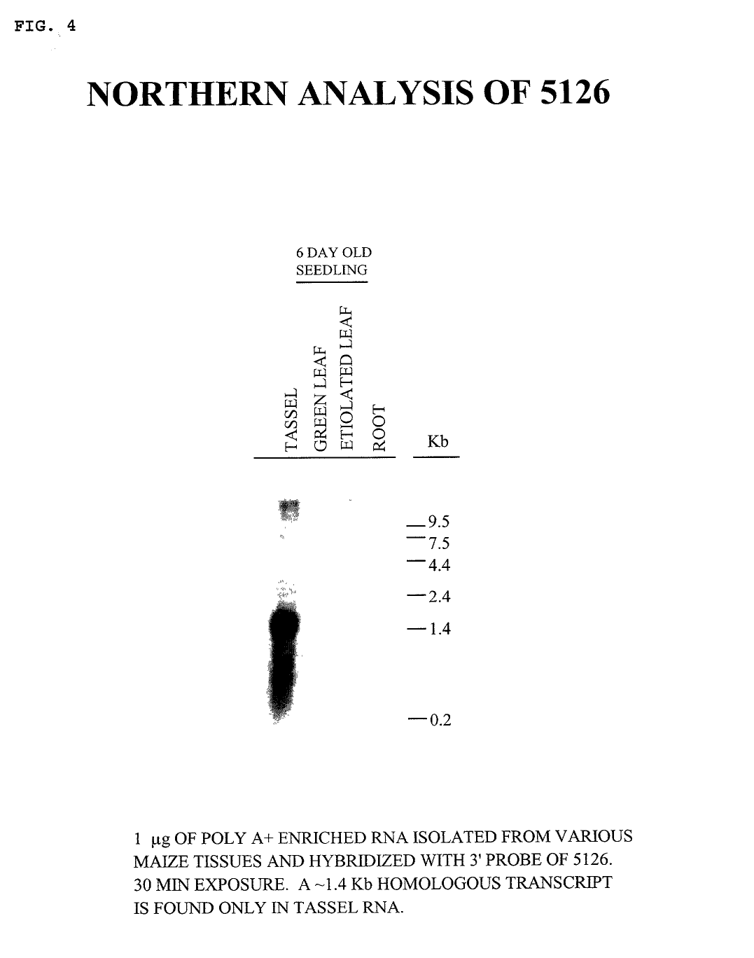

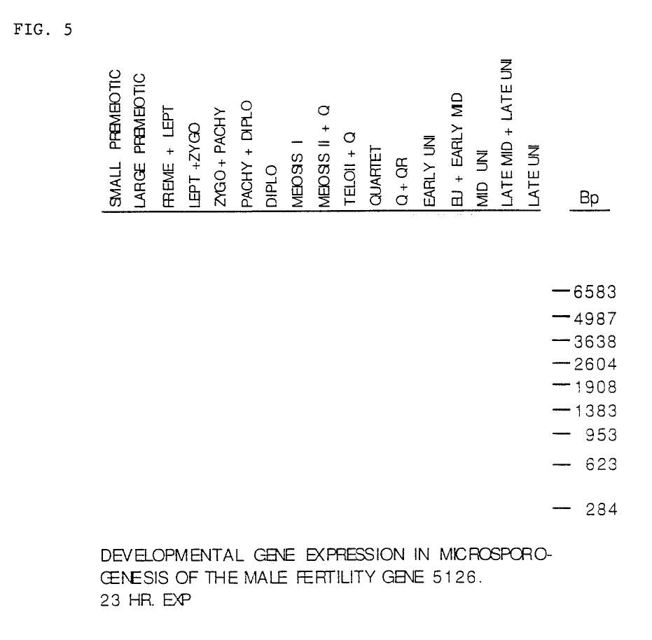

The hybridizing 3′ probe begins at nucleotide 896 of a clone, p5126-5. The clone has a length of 1.485 Kb that when compared to the transcript lengths in FIGS. 4 and 5 indicates that it represents a putatively full length cDNA. The 3′ probe was labeled with horseradish peroxidase using the Enhanced Chemiluminescence (ECL) system from Amersham Life Science, Inc. (Arlington Heights, Ill.). Hybridization of the probe and membrane washes followed the manufacturer's protocol for the ECL system described in ECL Direct™ Nucleic Acid Labeling and Detection Systems catalogue (1998) p. 114. The cDNA probe hybridized to transcripts approximately 1.4 Kb. The native transcript could be larger due to a longer polyA+tail.

The northern blot in FIG. 4 indicates that the native transcript is present only in mRNA from tassel tissue. In addition, the northern blot in FIG. 5 suggests that the maize Ms*5126 gene is transcribed as early as the meiosis/quartet stage with levels peaking at the early to early mid-uninucleate stages. The Ms*5126 signal rapidly drops off at stages later than early mid-uninucleate and is undetectable in late uninucleate microspores.

(3) Mutants Discovered by the Reverse Genetics Procedure:

Reverse Genetics technology permits rapid recovery of new alleles. Bensen, et al. 1995. The Plant Cell 7:75-84; Meeley, et al. 1995. Maize Genetics Cooperation Newsletter 69:67-82, each of which are hereby incorporated by reference. This rapid recovery can be accomplished by surveying a collection of DNA samples from individual plants, using a pair of polymerase chain reaction (PCR) primers. The pair of PCR primers can include, for example, one primer from the Terminal Inverted Repeat (TIR) region of a Mu element; and another primer could be from a putative Ms*5126-homologue clone. The purpose of the survey is to identify those individuals that produce a PCR product homologous to the cloned gene. Such products are a consequence of the Mu element insertion into the cloned gene. The cloned gene is confirmed as a Ms*5126 gene or a Ms*5126-homologue if a regenerated plant that possesses the DNA that yields the PCR product also segregates for male sterility.

For example, in the current invention, mutants were isolated using the reverse genetics procedure described above. A primer designated DO1967 (5′AGCAGCATGG ACACGACGAGTGAC3′) (SEQ ID NO:8) was synthesized using a maize Ms*5 126 cDNA sequence. The primer DO1967 was then paired with a primer designated DO938 (5°CCCTGAGCTC TTCGTCYATA ATGGCAATTA TCTC3′) (SEQ ID NO:9). The DO938 primer is a degenerate Mu primer homologous to the TIR regions of all characterized Mutator elements. The primer DO938 is also able to prime bi-directionally outward from either TIR region. PCR was performed with these two primers on 24,308 Mu-containing F1 maize individuals. Positive PCR reactions were detected by first blotting the reactions onto charged nylon membrane and then hybridizing the blots with a 3′ probe of Ms*5 126. Multiple positives were obtained from this screen. A particular F 1 maize mutant designated 54-D7 was chosen for further characterization. As shown on the Ms*5126 gene map in FIG. 6, a Mutator element was found in exon 2 of the Ms*5126 gene. The position of this insertion was confirmed by sequencing the DO1967-DO938 PCR product from the 54-D7 individual.

(a) Mutant Characterization:

F2 progeny from the mutant 54-D7 maize individual were analyzed by both PCR and DNA gel blot analysis. DNA from the F2 progeny was digested with HincII and hybridized with a 3′ probe of Ms*5126. The Ms*5126 (DO1967)/Mu-TIR (DO938) PCR product co-segregated with the 5.9 Kb HincII fragment. Plants homozygous for this fragment did not shed pollen. A total of 27 maize plants were analyzed.

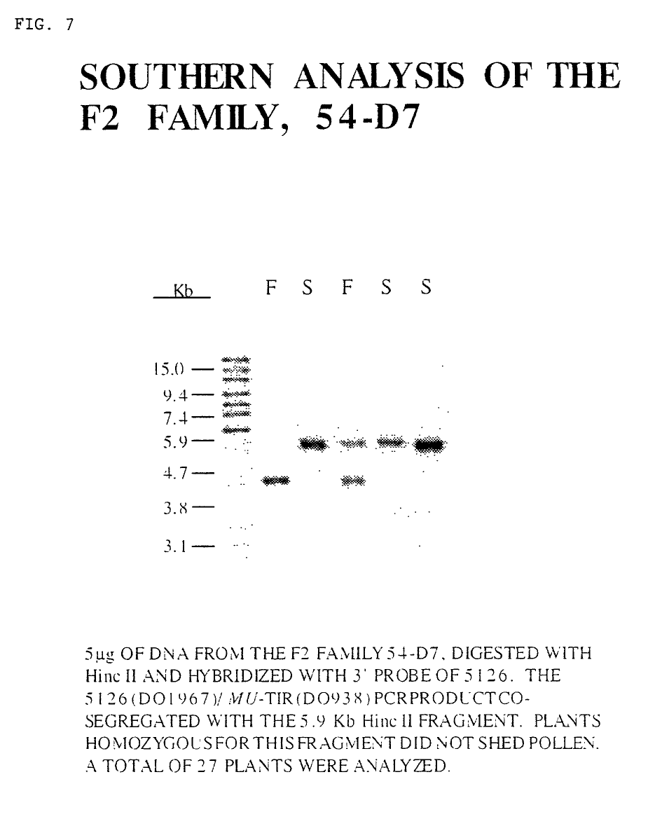

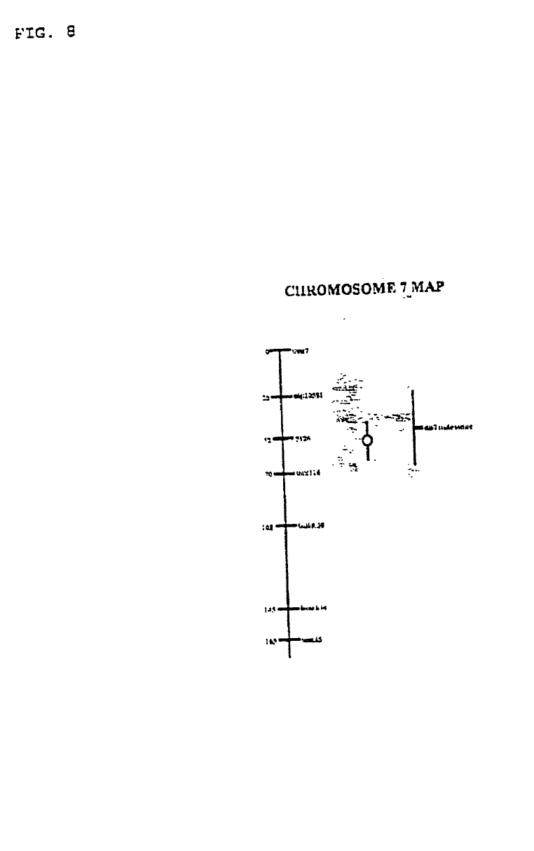

The southern blot of the 54-D7 F2 individuals, shown in FIG. 7, reveals a HincII polymorphism segregating in this family. Southern blots were performed by methods well known in the art, such as those disclosed in section 2.9.1 of Ausubel, et al., supra. The 5.9 Kb HincII fragment was found to co-segregate with DO1967-DO938 PCR positive maize plants. Tassels of these maize plants that were homozygous for this HincII fragment did not shed pollen. Although these plants would occasionally extrude anthers, no viable pollen could be obtained.

The microspore wall in 54-D7 mutants appears somewhat complete and the microspores occasionally progress into the starch engorgement stage of pollen development. The microspore cells were extruded from anthers and stained with acetocarmine. These were viewed under a microscope at magnifications of 16× to 40×. Anther squashes from mutant maize plants show a very low number of normal pollen grains with an unusually high number of pollen abortions when compared to wild type siblings. This suggests that a Ms*5126 gene product is required for normal pollen development. The few “normal looking” pollen grains found in the mutant anther squashes may be due to Mutator excision from the maize Ms*5126 gene, incomplete inactivation of the gene, or redundancies in gene function for microspore development.

(b) 54-D7 and ms7 Mutants are Distinguished:

The maize Ms*5126 gene was molecularly mapped close to the centromere on chromosome 7 of the maize genome. The male sterile locus ms7 has been genetically mapped near the maize Ms*5126 gene on chromosome 7. However, cytologic differences between 54-D7 and ms7 mutants suggest that the two mutations are not allelic. For example, the microspore wall is thin and poorly developed in the ms 7 mutation as compared to a more complete microspore wall formation in 54-D7 mutants. Additionally, ms7 microspores do not progress into the starch engorgement stage of pollen development as is occasionally the case with 54-D7 microspores. Most importantly, intercrosses between 54-D 7 and ms 7 mutants have confirmed that they are not allelic.

D. Impairing the Expression of a MS*5126 Gene

Gene expression can be inhibited through means well known in the art. Such means may include: (1) sequestering the mRNA that corresponds to a Ms*5126 gene with antisense sequences complementary to the mRNA, (2) introducing additional copies of the Ms*5126 gene into the genome of the host plant so that “co-suppression” results, (3) disrupting the Ms*5126 gene coding sequence via recombinant recombination, (4) disrupting the Ms*5126 gene coding sequence via insertion of transposable elements, (5) replacing the Ms*5126 gene with another genetic construct through gene targeting, or (6) inhibiting translation by cleaving the mRNA that corresponds to the Ms*5126 gene with ribozymes. Constructs containing all or part of the Ms*5126 gene can be reintroduced into plant cells by a variety of techniques including particle bombardment [Fitch, et al. 1990. Plant Cell Rep. 9:189] or Agrobacterium-mediated transformation [Fitch, et al. 1993. Plant Cell Tiss. Org. Cult. 32:205]. After introduction in a plant cell, these compositions and the constructs containing them can be used to control the expression of a Ms*5126 protein in maize or in other Ms*5126-homologues, such as in petunia, grape, tobacco, rice and pine.

(1) Antisense Inhibition of Gene Expression:

Antisense RNA transcripts have been used to suppress the expression of genes in plants. Suppression of gene expression in plants has been reported for several genes including: (1) the tomato polygalacturonase gene [Smith, et al. 1988. Nature 334:724 and U.S. Pat. No. 5,107,065, the disclosure of which is herein incorporated by reference]; (2) the tomato ACC synthase gene, LE-ACS2 [Oeller, et al. 1991. Science 254:437; (3) the Brassica stearoyl-acyl-carrier protein desaturase gene [Knutzon, et al 1992. Proc. Natl. Acad. Sci. (USA) 89:2624-2628]; (4) the petunia chalcone synthase gene [van der Krol, et al. 1988. Nature 333: 866-869]; (5) the potato granule-bound starch synthase gene [Visser, et al. 1991. Mol. and Gen. Genet. 225:289-296]; and (6) the tomato pectin methylesterase gene [Tieman, et al. 1992. Plant Cell 4:667-679].

Antisense inhibition involves sequestering the mRNA corresponding to a Ms*5126 gene with an antisense oligonucleotide or nucleotide analogue complementary to the sequence of the mRNA. While not limiting the invention to any particular theory, it is believed that the antisense transcripts form a duplex with the sense transcripts thereby preventing the splicing, transcription or translation of the sense RNA transcript.

To produce an antisense Ms*5126 RNA transcript, gene sequences derived from a Ms*5126 gene are placed downstream of a promoter in the opposite transcriptional orientation. The “opposite transcriptional orientation” is relative to the direction of transcription of the endogenous Ms*5126 gene. For example, a Ms*5126 protein is produced when the Ms*5126 gene is placed in an expression construct in the same transcriptional orientation relative to an active promoter. However, when the gene is placed in the opposite orientation relative to the suitable promoter, antisense sequences are produced.

The molecules used to form the construct may have a variety of chemical constitutions, so long as they retain the ability specifically to bind at the indicated control elements. Especially preferred molecules are oligo-DNA, RNA and protein nucleic acids (PNAs). The oligonucleotides of the present invention can be based, for example, upon ribonucleotide or deoxyribonucleotide monomers linked by phosphodiester bonds, or by analogues linked by methyl phosphonate, phosphorothioate, or other bonds. These can be engineered using standard synthetic techniques to specifically bind the targeted control region(s). Oligonucleotides that are nuclease resistance such as phosphorothioate or methyl phosphonate-linked analogues are preferred over phosphodiester-linked oligonucleotides that are particularly susceptible to nucleases. Stein, et al. 1988. Oligodeoxynucleotides-Antisense Inhibitors of Gene Expression London: McMillan Press. Other linkages may be selected for use in the present invention. Oligonucleotides may be prepared by methods well-known in the art, for instance using commercially available machines and reagents available from Perkin-Elmer/Applied Biosystems (Foster City, Calif.). Antisense molecules should be small in order to be highly specific to the Ms*5126 mRNA. Molecules that correspond to less than about 50 nucleotides are preferred. The resulting antisense construct is introduced into a plant cell host such that the antisense construct directs the transcription of antisense RNA transcripts. Introduction of an antisense construct is achieved through methods known in the art. The section entitled “Transformation of Plant Cells,” infra, provides a more comprehensive discussion of methods for inserting antisense constructs into a plant genome.

(2) Co-Suppression of Gene Expression:

Another approach in impairing the expression of a Ms*5126 gene is through the “co-suppression” technique. Co-suppression occurs when one or more copies of a gene, or one or more copies of a substantially similar gene, are introduced into a cell's genome. Via an unknown trans mechanism, loss of function of the endogenous gene will sometimes occur. Introduction of a transgene or gene construct is achieved through methods known in the art, such as those discussed in “Transformation of Plant Cells,” infra.

Co-suppression of a number of plant genes has been reported, for example, such as with the petunia dihydroflavonol-4-reductase gene. See van der Krol, et al. 1990. Plant Cell 2:291. In addition, U.S. Pat. No. 5,283,184 describes the co-suppression of the endogenous chalcone synthase gene in petunia and in chrysanthemum. Co-suppression occurred when an exogenous transgene comprising a chimeric gene encoding chalcone synthase was introduced into the cell. The disclosure of U.S. Pat. No. 5,283,184 is incorporated by reference in its entirety.

Co-suppression is a form of homology-dependent gene silencing. Matzke, et al. 1995. Plant Physiol. 107:679. Co-suppression may involve the coordinate repression (silencing) of a transgene and a homologous endogenous gene or the repression of two homologous transgenes. While the invention is not limited to a particular theory, it is believed that co-suppression may involve post-transcriptional events, such as the induction of RNA degradation by the over-expression of a given transcript due to expression of both the endogenous RNA and the transgene RNA transcripts. Alternatively, the interaction of the transgene and the endogenous gene may occur on a DNA-DNA level resulting in the methylation of the gene sequences. Methylated gene sequences are often transcriptionally inactive in plants. Regardless of the exact mechanism, the introduction of a transgene capable of expressing sense Ms*5126 transcripts can be used to inhibit expression of the Ms*5126 gene in maize and Ms*5126-homologues in other crops.

(3) Inhibition of Gene Expression via Homologous Recombination:

Expression of a Ms*5126 gene can be disrupted via homologous recombination. Homologous recombination can be used to stably integrate a vector into the Ms*5126 coding sequence of the host chromosome. Transformation of a plant cell with a vector is achieved through methods known in the art, such as those described in the section below entitled “Transformation of Plant Cells.” Homologous recombination is a rare event. Therefore, in order to select for the small number of transformants that may result, a targeting vector designed to introduce an antibiotic resistance gene that is normally lacking in the target cells is required. Integration of the antibiotic resistance gene within the coding sequence of the Ms*5126 gene also serves to disrupt normal transcription of the gene and produces an aberrant, non-functional Ms*5126 protein product.

(4) Inhibition of Gene Expression via Transposable Elements:

Another method of inhibiting expression of a Ms*5126 gene is to introduce insertions into the Ms*5126 coding sequence via naturally occurring transposable elements. Transposable elements are capable of transposition when introduced into unrelated plant species. Vedel, et al. 1994. Plant Physio. Biochem. 32:607; Walbot, et al. 1992. Annu. Rev. Plant Physio. Plant Mol. Biol. 43:49-82. An introduction of transposable elements into plant cells is performed through methods known in the art. The section below, entitled “Transformation of the Plant Cell,” provides a comprehensive discussion of such methods.

In plants, the Mutator (Mu) transposable element system has been used to clone many genes. Walbot, et al., supra at pp. 49-82. To confirm that a tagged gene has been isolated, reverse genetics technology may be employed to permit rapid recovery of new alleles containing Mu insertions. Bensen, et al. supra at pp. 75-84. Moreover, PCR can also be used to detect an insertion event within a particular gene of interest. Ballinger et al. 1989. Proc. Natl. Acad. Sci. 86:9402-06; Kaiser et al. 1990. Proc. Natl. Acad. Sci. 87:1686-90; Zwaal et al. 1993. Proc. Natl. Acad. Sci. 90:7431-7435. Performing the PCR procedure would involve using two oligonucleotide primers with one primer complementary to a sequence within a particular gene of interest, and the other primer complementary to a portion of the TIR sequence of the transposable element. For example, in accordance to the present invention, PCR was performed in order to isolate F1 individuals possessing a Mu insertion within the maize Ms*5126 gene sequence. The section entitled “Mutants discovered by the reverse genetics procedure,” supra, provides a more detailed description of the experimental procedure.

Aarts et. al. isolated a sterile male mutant in A. thaliana by insertional mutagenesis using a maize Enhancer-Inhibitor transposition system. The authors prepared a two-element vector containing a non-mobile En transposase source under the control of a CaMV35S promoter, and used a mobile I element as an insertion mutagen. This construct was introduced into A. thaliana by transformation with Agrobacterium tumefaciens. Hygromycin resistance that was conferred by a hygromycin phosphotransferase gene was fused with the two element vector. This antibiotic resistance allowed for the selection of primary transformants. A male sterile plant was found in the third generation of selfed progeny. Aarts et. al. 1993. Nature 363:715-717. In a related context, in U.S. Pat. No. 5,850,014, male sterility is achieved when a gene affecting fertility is inactivated and replaced with a genetically-engineered gene that is linked to an inducible promoter. Male fertility is restored by inducing expression of the gene via the promoter.

(5) Inhibition of Gene Expression via mRNA Splicing with Ribozymes:

Another means to impair the expression of a Ms*5126 gene is to target the Ms*5126 genomic region using ribozymes. Ribozymes are synthetic RNA molecules which comprise a hybridizing region that is complementary to two regions. Each region should be at least within 5 contiguous nucleotide bases from the target sense mRNA. Ribozymes possess highly specific endoribonuclease activity that automatically cleaves the target mRNA. A complete description of the function of ribozymes is contained in PCT Application WO89/05852. Insertion of a ribozyme construct into a plant cell is achieved through methods known in the art, such as those described in “Transformation of Plant Cells,” infra.

The present invention provides a ribozyme molecule comprising of at least 5 contiguous nucleotide bases. The molecule should be able to form a hydrogen-bonded complex with a sense mRNA transcription product of a Ms*5126 gene or a Ms*5126-homologue. Although preferred ribozyme molecules hybridize to at least about 10 or 20 nucleotides of the target molecules, the present invention extends to molecules capable of hybridizing to at least about 50 to 100 nucleotide bases in length, or a molecule capable of hybridizing to a full-length or substantially full-length mRNA transcriptions product of a Ms*5126 gene or a homologue. Inhibition of gene expression is accomplished when ribozyme molecules hybridize to the targeted sense MRNA and cleave it so that it can no longer be translated to synthesize a functional polypeptide product.

(6) Inhibition of Gene Expression via Gene Replacement/Gene Targeting:

In another embodiment of the invention, the sequence of a Ms*5126 gene is disrupted via gene targeting. At least part of a Ms*5126 gene or a Ms*5126-homologue may be introduced into target cells containing an endogenous Ms*5126 gene. Introduction of a gene construct is achieved through methods known in the art, such as those described in “Transformation of Plant Cells,” infra. The introduced nucleic acid molecule may comprise a missense or non-sense mutation relative to the corresponding sequence in the endogenous gene. Once the nucleic acid molecule is introduced, it hybridizes and replaces the corresponding sequence such that the form and/or function of the endogenous Ms*5126 gene is altered. Consequently, the resulting Ms*5126 protein possesses catalytic activity, substrate affinity or other polypeptide function that is different from the endogenous Ms*5126 protein. The altered Ms*5126 protein expression is manifested as a phenotype of male sterility.

(7) Transformation of Plant Cells:

Approaches to introducing recombinant DNA that carry a sense, antisense, gene-targeting, ribozyme or co-suppression molecule into plant tissue include, but are not limited to, direct DNA uptake into protoplasts (Krens, et al. 1982. Nature 296:72-74); PEG-mediated uptake to protoplasts (Armstrong, et al. 1990. Plant Cell Reports 9:335-339); microparticle bombardment electroporations (Fromm, et al. 1985. Proc. Natl. Acad. Sci. (USA) 82:5824-5828); microinjections of DNA (Crossway, et. al. 1986. Mol Gen. Genet. 202:179-185); microparticle bombardment of tissue explants or cells (Christou, et al. 1988. Plant Physiol 87:671-674); vacuum-infiltration of plant tissue with nucleic acid, in planta transformation (Chang, et al. 1994. The Plant Journal 5:551-558); or T-DNA mediated transfer from Agrobacterium to the plant tissue. Representative T-DNA vector systems are described in the following references: An, et al. 1986. EMBO J. 4:277-284; Herrera-Estrella, et al. 1983. Nature 303:209-213; Herrera-Estrella, et al. 1983, EMBO J. 2: 987-995; Herrera-Estrella, et al. 1985. Plant Genetic Engineering, New York: Cambridge University Press, pp. 63-93. Microparticle bombardment of cells calls for a microparticle to be propelled into a plant cell. Any suitable ballistic cell transformation methodology and apparatus can be used in practicing the present invention. Exemplary apparatus and procedures are disclosed in U.S. Pat. No. 5,122,466, and in U.S. Pat. No. 4,945,050. When using ballistic transformation procedures, the genetic construct may incorporate a plasmid capable of replicating in the cell to be transformed. Examples of microparticles that are suitable for use in such systems include 1 to 5 μm gold spheres. The DNA construct may be deposited on the microparticle by any suitable technique, such as by precipitation.

Cells from any plant tissue capable of subsequent clonal propagation, whether by organogenesis or embryogenesis, may be transformed with a ribozyme, antisense, gene-targeting or co-suppression molecule. The term “organogenesis” means a process by which shoots and roots are developed sequentially from meristematic centers. The term “embryogenesis” means a process by which shoots and roots develop together either from somatic cells or from gametes in a concerted but unsequential fashion.

The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g. apical meristem, axillary buds, and root meristems) and induced meristem tissue (e.g. cotyledon meristem and hypocotyl meristem).

(8) Hybridization under Stringent Conditions:

An aspect of the present invention provides a method of expressing in a plant a sense, antisense, ribozyme, gene-targeting or co-suppression molecule made up of a sequence of nucleotides capable of hybridizing under at least low stringency conditions to at least 20 contiguous nucleotides in SEQ ID NO: 1. It is understood in the art that certain modifications, including nucleotide substitutions amongst others, may be made to the molecules of the present invention, without destroying the efficacy of the molecules in inhibiting the expression of a Ms*5126 gene. It is therefore within the scope of the present invention to include any nucleotide sequence variants, homologues, analogues, or fragments of a Ms*5126 gene encoding the same. The only requirement is that the nucleotide sequence variant, when transcribed, should produce a genetic construct that is capable of hybridizing to a sense MRNA molecule.

For the purposes of defining the level of stringency, reference can be made to Maniatis, et al. at pages 387-389 which are incorporated herein by reference. Maniatis, et al. 1982. Molecular Cloning: A Laboratory Manual. Cold Spring Harbor: Cold Spring Harbour Laboratories. pp. 387-389. A high stringency wash, for example, can be 0.1-0.2× SSC, 0.1% (w/v) SDS at 55-65° C. for 20 minutes and a low level of stringency wash, for example, can be 2× SSC, 0.1-0.5% (w/v) SDS at >45° C. for 20 minutes. The hybridization temperature, which depends on the G +C content of the DNA, is chosen so as to minimize the formation of DNA·DNA hybrids while allowing DNA·RNA hybrids to form. The following table from Maniatis, et al., supra at 208, gives the approximate hybridization temperatures for DNAs of different G+C content. Alternative conditions would pertain, depending on concentration, purity, and source of nucleic acid molecules.

| G + C Content | Hybridization Temperature |
|---|---|
| 41% | 49° C. |
| 49% | 52° C. |
| 58% | 60° C. |

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization membrane or on the type of hybridization probe used. For the purposes of clarification of the parameters affecting hybridization between nucleic acid molecules, reference is found in section 2.10.8-2.10.16 of Ausubel et al., supra, which is herein incorporated by reference.

(9) Promoters:

Placing a genetic construct under the regulatory control of a promoter sequences means positioning the molecule such that expression is controlled by the promoter sequence. Promoters are generally positioned 5′ (upstream) to the genes that they control. In the construction of heterologous promoter/structural gene combinations it is generally preferred to position the promoter at a distance from the gene transcription start site that is approximately the same as the natural distance between the promoter and the gene it controls, i.e. the gene from which the promoter is derived. As is known in the art, some variation in the distance can be accommodated without loss of promoter function. Similarly, the preferred positioning of a regulatory sequence element with respect to a heterologous gene to be placed under its control is defined by the positioning of the element in its natural setting, i.e. the genes from which it is derived. Again, as is known in the art, some variation in this distance can also occur. Preferred promoters may contain additional copies of one or more specific regulatory elements. This may further enhance expression of the inserted construct and/or to alter the spatial expression and/or temporal expression of the construct.

A preferred promoter includes a tissue-specific or cell-specific promoter that controls gene expression in cells that are critical to the formation or function of pollen. Such cells would include tapetal cells, pollen mother cells and early micropores. An “anther specific promoter” is a DNA sequence that directs a higher level of transcription of an associated gene in anther tissue than in some or all other tissues in a plant. Preferably, the promoter only directs expression in anthers. The anther-specific promoter of a gene directs the expression of a gene in anther tissue but not in other tissues, such as root or coleoptile. Promoters of this specificity are described, for example, in published European application 93810455.1, the contents of which are hereby incorporated by reference. Examples of suitable promoters are G9, SGB6, TA39 and the Ms*5126 promoter described in U.S. Pat. No. 5,750,868.

E. Regeneration of Transformed Plants

(1) Regeneration Protocols:

In seed plants, two pathways lead to regeneration: (1) the formation of adventitious shoots followed by rooting of cuttings or by grafting; or (2) the formation of embryo-like structures that are developed directly into plants. Methods of regenerating a plant from a transformed cell or culture vary according to the plant species but should comprise of methods known in the art. Growth conditions for embryo and shoot formation for various species of monocotyledons and dicotyledons can be found by referring to Binding, H., supra at 21-73.

(2) Regeneration of Maize Plants:

Fertile transgenic maize plants can be regenerated by transferring embryogenic callus to MS medium containing 0.25 mg/L 2,4-D and 10.0 mg/L 6-benzylaminopurine. Tissue should be maintained on this medium for approximately 2 weeks and subsequently transferred to MS medium without hormones. Shillito, et al. 1989. Bio/Technol. 7:581-587. Shoots that developed after 2-4 weeks on hormone-free medium should then be transferred to MS medium containing 1% sucrose and solidified with 2 g/L Gelgro® in Plant Con® containers where rooting occurred. Alternative regeneration routes using media containing high cytokinin/auxin ratios are also successful. More mature embryonic callus can be regenerated on N6 medium (Chu, et al. 1975. Sci. Sin. 18: 659-668) containing 6% sucrose and no hormones (Armstrong, et al. 1985. Planta 164:207-214) for 2 weeks, followed by transfer to MS medium without hormones as described above. Regeneration should be performed at 25° C. under fluorescent lights (250 μE m⁻² sec⁻¹). After approximately 2 weeks, developing plantlets should then be transferred to soil, hardened in a growth chamber (85% relative humidity, 660 ppm CO₂ 350 μE m⁻² sec⁻¹) and grown to maturity in either a growth chamber or the greenhouse. Gordon-Kamm, et al. 1990. Plant Cell 2:603-618.

An alternate method for regeneration of transformed maize cells can be found in Register, et al. 1994. Plant Mol. Biol. 25:951-961. Maize plants are regenerated by transferring tissue to MS-based medium containing 1 g/l myo-inositol, 1 mg/l NAA, 6% (w/v) sucrose, and 0.3% (w/v) Gelrite pH 6.0. After 2-3 weeks, the tissue can then be transferred to MS medium containing 0.25 mg/l NAA and 3% (w/v) sucrose and placed in the light, where embryo germination occurs. Plantlets are then grown in half-strength MS-based medium containing 500 mg/l myo-inositol, 3% (w/v) sucrose, and 0.3% Gelrite pH 6.0 for about 1-2 weeks prior to transfer to the greenhouse.

(3) Regeneration of Rice Plants:

Rice plants are regenerated by means known to those of skill in the art. The following steps exemplify a transformation and regeneration protocol for rice: The bacterial hph gene encoding hygromycin B resistance (Hm^{r}) was introduced into protoplasts of Oryza sativa by electroporation. After 2-3 weeks of selection with hygromycin B (20 μg ml⁻¹), resistant colonies became clearly visible. Most of the Hm^{r} colonies continued to grow after transferring them to a solid N6 medium containing the same concentration of hygromycin B. After 3-4 weeks, shoots as well as roots became visible. Plantlets regenerated from Hm^{r} calli were transferred to pots where they grew to maturity, flowered and set seeds. Shimamoto, et al. 1989. Nature 338:274-276.

F. Method of Obtaining Homologues of a Ms*5126 gene in Other Plant Species

Ms*5126 homologues can be readily obtained from a wide variety of plants species by cloning methods known in the art. Such methods could include screening a cDNA or genomic library with a probe that specifically hybridizes to a native Ms*5126 sequence under stringent conditions, or by PCR or other amplification method using a primer or primers that specifically hybridizes to a native Ms*5126 sequence under stringent conditions.

Homologues of the maize Ms*5126 gene have been discovered in such plant species as Petunia hybrida, grape (Vitis cv. optima), tobacco (Nicotiana sylvestris), pine (pinus radiata) and rice (O. sativa). Homologues of the maize Ms*5126 gene can be found by following the methodology used to isolate homologues of the HY4 gene (WO 96/01897) in other plant species. To isolate a homologous Ms*5126 gene in other species, cDNA clones should be isolated by screening the specie specific cDNA library using standard methods, such as those found in section 6 of Ausubel, et al., supra. Sequence identity analysis will determine those genes that are highly conserved among different plant species and which, therefore, are homologous to the maize Ms*5126 gene.

All publications and patent applications referred to in this specification are indicative of the level of skill of those in the art to which the invention pertains. All publications and patent applications are herein incorporated by reference to the same extent as if each individual publications or patent applications were specifically and individually indicated to be incorporated by reference in its entirety.

Other objects, features and advantages of the present invention will become apparent from the foregoing detailed description and examples. It should be understood, however, that the detailed description and the specific examples, while indicating preferred embodiments of the invention, are given only by way of illustration.

What is claimed is:

1. A plant that comprises an Ms*5126 gene, the expression of which isaltered by human intervention such that male fertility in said plant is altered.

2. A method for affecting male fertility in a plant, comprising the steps of (A) modifying plant material to alter the expression of the endogenous Ms*5126 gene and; (B) obtaining from said material a plant that comprises the alter endogenous Ms*5126 gene such that a phenotype of male sterility is expressed.

3. A method of altering male fertility in a plant, comprising altering an Ms*5126 gene, such that male fertility in said plant is altered.
